(19) United States
(12) Patent Application Publication
(10) Pub. No.: US 2026/0248378 A1
(43) Pub. Date: Aug. 27, 2026

(54) VISION CORRECTION SIMULATION TOOL

(71) Applicant: Johnson & Johnson Vision Care, Inc., Jacksonville, FL (US)

(72) Inventors: Khaled Chehab, Jacksonville, FL (US); Thomas R Karkkainen, Orange Park, FL (US); Derek Nankivil, Jacksonville, FL (US); Andrew Paul Frank Milton, Cambridge (GB); Richard Thomas Bown, Cambridge (GB)

(21) Appl. No.: 19/325,335

(22) Filed: Sep. 10, 2025

Related U.S. Application Data
(60) Provisional application No. 63/693,857, filed on Sep. 12, 2024.

(51) Int. Cl. A61B 3/00; A61F 2/16; G06T 5/50; G06T 5/70; G06T 11/60; G16H 40/63

(52) U.S. Cl. CPC A61B 3/0025; A61F 2/16; G06T 5/50; G06T 5/70; G06T 11/60; G16H 40/63; G06T 2207/20212; G06T 2207/30041; G06T 2210/41

(57) ABSTRACT

In one general aspect, methods may include receiving a plurality of image sets. At least one of the image sets may include a set of versions. At least one of the set of versions may correspond to at least one optical attribute of a contact lens, interocular lens (IOL), and/or progressive addition lens (PAL). The at least one of the set of versions may include an image pair. The image pair may include a left eye image and a right eye image. Methods may include receiving an indication of contact lens, IOL, or PAL information. The contact lens, IOL, or PAL information may include the at least one optical attribute. Methods may include causing the left eye image to be presented to a left eye of the user. Methods may include causing the right eye image to be presented to a right eye of the user.

### BACKGROUND

Typically, when a user is assessed for suitability to a mode of vision correction fit such as a contact lens (CL), interocular lens (IOL), and/or progressive addition lens (PAL) fit, the assessment is performed in one environment. For example, the assessment may be performed in a well-lit environment, such as in an office. However, the user may use the vision correction in environments that differ from the one environment.

Improvements are needed.

### SUMMARY

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

In one general aspect, methods may include receiving a first image which may have at least a first text. Methods may include receiving a second image which may have at least a second text. Methods may include generating, based at least on the first image, at least one background image by applying a first blurring effect to at least a portion of the first image.

The background image may be generated to emulate a distance vision environment. Methods may include generating, based at least on the second image, at least one foreground image by applying a second blurring effect to at least a portion of the second image. The foreground image may be generated to emulate a near vision environment. Methods may include generating a right eye image associated with at least one optical attribute of a contact lens, IOL, or PAL. The right eye image may include at least a first portion of the foreground image overlaying at least a first portion of the background image. The right eye image may be configured to be presented to a right eye of a user. Methods may also include generating a left eye image associated with the at least one optical attribute of the contact lens, IOL, or PAL, where the left eye image may include at least a second portion of the foreground image overlaying at least a second portion of the background image, where the left eye image is configured to be presented to a left eye of the user while the right eye image is presented to the right eye of the user. Other embodiments of this aspect include corresponding computer systems, apparatus, and computer programs recorded on one or more computer storage devices, each configured to perform the actions of the methods.

In one general aspect, methods may include receiving a plurality of image sets. At least one of the image sets may include a set of versions of one or more images. As an example, version may include similar images with areas or characteristics adapted between them. At least one of the set of versions may correspond to at least one optical attribute of a contact lens, IOL, or PAL. The at least one of the set of versions may include an image pair. The image pair may include a left eye image and a right eye image. Methods may include receiving an indication of contact lens, IOL, or PAL information. The contact lens, IOL, or PAL information may include the at least one optical attribute. Methods may include causing the left eye image to be presented to a left eye of a user. Methods may include causing the right eye image to be presented to a right eye of the user. Other embodiments of this aspect include corresponding computer systems, apparatus, and computer programs recorded on one or more computer storage devices, each configured to perform the actions of the methods.

In one general aspect, methods may include receiving at least one optical attribute (e.g., an indication or representation of at least one optical attribute) of a multifocal contact lens, IOL or PAL. Methods may include presenting a left eye image to a left eye of a user. The left eye image may emulate vision associated with the at least one optical attribute. Methods may include presenting a right eye image to a right eye of the user. The right eye image may emulate the vision associated with the at least one optical attribute. Methods may include receiving a satisfaction score of the vision associated with the at least one optical attribute from the user. Methods may include predicting a likelihood of satisfaction with multifocal contact lenses, IOL, or PAL having the at least one optical attribute based on the satisfaction score. Other embodiments of this aspect include corresponding computer systems, apparatus, and computer programs recorded on one or more computer storage devices, each configured to perform the actions of the methods.

In one general aspect, systems may include one or more processors configured to: receive a first image which may have at least a first text; receive a second image which may have at least a second text; generate, based at least on the first image, at least one background image by applying a first blurring effect to at least a portion of the first image, where the background image is generated to emulate a distance vision environment; generate, based at least on the second image, at least one foreground image by applying a second blurring effect to at least a portion of the second image, where the foreground image is generated to emulate a near vision environment; generate a right eye image associated with at least one optical attribute of a contact lens, IOL, or PAL, where the right eye image may include at least a first portion of the foreground image overlaying at least a first portion of the background image, where the right eye image is configured to be presented to a right eye of a user; and generate a left eye image associated with the at least one optical attribute of the contact lens, IOL, or PAL, where the left eye image may include at least a second portion of the foreground image overlaying at least a second portion of the background image, where the left eye image is configured to be presented to a left eye of the user while the right eye image is presented to the right eye of the user. Other embodiments of this aspect include corresponding computer systems, apparatus, and computer programs recorded on one or more computer storage devices, each configured to perform the actions of the methods of the present disclosure.

In one general aspect, systems may include one or more processors configured to: receive a plurality of image sets, where at least one of the image sets may include a set of versions, where at least one of the set of versions corresponds to at least one optical attribute of a contact lens, IOL, or PAL, where the at least one of the set of versions of images may include an image pair, and where the image pair may include a left eye image and a right eye image; receive an indication of contact lens, IOL, or PAL information, where the contact lens, IOL, or PAL information may include the at least one optical attribute; cause the left eye image to be presented to a left eye of a user; and cause the right eye image to be presented to a right eye of the user. Other embodiments of this aspect include corresponding computer systems, apparatus, and computer programs recorded on one or more computer storage devices, each configured to perform the actions of the methods.

In one general aspect, systems may include one or more processors configured to: receive an indication of at least one optical attribute; present a left eye image to a left eye of a user, where the left eye image emulates vision associated with the at least one optical attribute; present a right eye image to a right eye of the user, where the right eye image emulates the vision associated with the at least one optical attribute; receive a satisfaction score of the vision associated with the at least one optical attribute from the user; and predict a likelihood of satisfaction with multifocal contact lenses, IOLs, or PALs having the at least one optical attribute based on the satisfaction score. Other embodiments of this aspect include corresponding computer systems, apparatus, and computer programs recorded on one or more computer storage devices, each configured to perform the actions of the methods.

These and other features and advantages are described in greater detail below.

### BRIEF DESCRIPTION OF THE DRAWINGS

Some features are shown by way of example, and not by limitation, in the accompanying drawings. In the drawings, like numerals reference similar elements.

FIG. 1 shows an example image according to the systems and methods described herein.

FIG. 2 shows an example left eye image and right eye image pair according to the systems and methods described herein.

FIG. 3 shows an example head-mount according to the systems and methods described herein.

FIG. 4 shows example trial frames according to the systems and methods described herein.

FIG. 5 shows an example tunnel according to the methods and systems described herein.

FIG. 6 shows an example display according to the systems and methods described herein.

FIG. 7 shows an example display holder according to the systems and methods described herein.

FIG. 8 shows an example device according to the systems and methods described herein.

FIG. 9 shows an example front view of the head-mount and an example front view of the left eye image and the right eye image.

FIG. 10 shows a flow diagram of an example method described herein.

FIG. 11 shows a flow diagram of an example method described herein.

FIG. 12 shows a flow diagram of an example method described herein.

The accompanying drawings show examples of the disclosure. It is to be understood that the examples shown in the drawings and/or discussed herein are non-exclusive and that there are other examples of how the disclosure may be practiced.

### DETAILED DESCRIPTION

The accompanying drawings, which form a part hereof, show examples of the disclosure. It is to be understood that the examples shown in the drawings and/or discussed herein are non-exclusive and that there are other examples of how the disclosure may be practiced.

It is to be understood that both the following general description and the following detailed description are exemplary and explanatory only and are not restrictive. Methods and systems for assessing suitability for a vision correction including contact lenses, PALs or IOLs are described.

As people age, the eye becomes less able to accommodate and focus at near. The loss of accommodation with age is known as presbyopia. The average onset age of presbyopia is around 42 and everyone is affected by presbyopia by the age of 55. Multifocal contact lenses (MFCLs) are often used to give presbyopes functional vision for near, intermediate and distant objects. There are about 2 billion presbyopes around the world, yet MFCL penetration is below 5%. Also, approximately 5 million contact lens wearers aged 40 and above drop out of contact lenses annually. The lack of penetration of MFCLs is driven by several factors including:
Financial incentives motivate eye care practitioners (ECPs) to prescribe progressive spectacles over MFCLs.
Fitting of MFCLs sometimes consumes too much chair time.
Success with MFCLs is perceived as low.
People (e.g., users, subjects, patients, contact wearers, etc.) are not aware of MFCLs and are not familiar with the vision that MFCLs provide.

The factors listed above diminish a person's opportunity and motivation to try MFCLs.

In many instances, an ECP defers initiating conversations about aging and presbyopia until a person has symptoms. Hence, 40+ year old contact lens wearers are likely to stop using contact lenses prior to trying MFCLs. In another situation, people are not aware that MFCLs exist or can address distance and near vision symptoms. Moreover, managing expectations is an art and the approach varies between ECPs. The ECP skills in managing expectations has a positive or negative influence on the breadth of potential candidates that may opt in to try MFCLs as an option for presbyopia correction.

Further, experience in fitting MFCLs varies tremendously between ECPs. Hence, capability or lack of capability and experience in fitting MFCLs has a significant or perceived impact on chair time. Thus, a substantial proportion of ECP's elect not to offer MFCLs as an option for their contact lens wearing population due to lack of confidence in the outcome, or due to fear of negative reputation if users are dissatisfied with the outcome. Thus, many ECPs prefer to offer other solutions such as reading glasses or Progressive Addition Lenses (PALs) to address users near vision needs.

This begs the question, how do ECPs determine patient suitability for a presbyopic correction? How do the ECPs determine which option is best for each user? Currently, ECPs typically ask about lifestyle and profession in an effort to assess motivations, personality, and visual demands to make a recommendation on the mode of correction-contact lens, PAL, or reading glasses. Typically, the assessment is performed in one environment. For example, the assessment may be performed in a well-lit environment, such as in an office. However, the user will wear the vision correction in environments that differ from the ECP office.

Thus, innovations are needed to improvement the process of determining presbyopic correction for each user. Improvements are needed to accurately identify a suitable vision correction and modality for a person. Successful systems and methods with these capabilities will raise awareness about presbyopic corrections, better match expectations with real-world experiences, reduce chair time needed to fit presbyopic corrections, and increase a success rate of presbyopic corrections.

A vision simulator and methods to simulate an expected real-world vision experience of an optical correction can help eye care practitioners guide users to a suitable presbyopic correction. The vision simulator causes a user to view an object that is blurred to simulate the vision experience with a particular form of optical correction (MFCL's, monovision, etc.). The user can then assess the vision quality of images that have been convolved with a point spread function (PSF) to emulate real-world vision experience for that particular form of optical correction. Vision quality can be assessed in real-time with questionnaires or psychophysical measurements where users provide a series of responses. Optical correction is not limited to multifocal vision but may include any optical correction including monovision, modified monovision, or single vision, reading glasses, and astigmatic correction. The simulated optical correction may also be generated either with or without a filter that operates on the amplitude of the pupil function either spectrally (spectral filter), spatially (apodization filter), or spatio-spectrally (spectral apodization filter).

The vision simulator improves the process of determining a suitable presbyopic correction for each user by providing the user with an understanding of presbyopia and its impact on far, intermediate, and near vision performance, and of the benefits of MFCLs for correction of vision at far, intermediate (computer distance), and near distances.

For the ECP, the vision simulator may improve fitting efficiency, helping the ECP to find a right fit quicker, resulting in increased fit success rate, reduced chair time, and reduced number of user revisits.

The present disclosure relates to systems and methods for assessing the simulated vision of contact lens, IOLs, or PALs are described. The present disclosure relates generally to assessing the simulated vision quality of contact lenses, IOLs, or PALs for a user by presenting a user with a left eye image and a right eye image that simulate wearing of the contact lenses, IOLs, or PALs.

As an illustrative example, an object in an image is blurred to simulate the vision experience of a patient (subject, user, contact wearer, person, etc.), corrected for distance vision, via a simulator. The user may assess the vision quality of convolved images that emulate the real-world vision experience of a user wearing an optical correction. The optical correction is not limited to multifocal vision, but may include any optical correction form including monovision, modified monovision, or single vision with or without a filter including reading glasses and astigmatic correction.

As an example, the simulator may comprise a viewing tunnel having two independent channels, one for each eye. This allows for the presentation of any or all the multifocal prescription (Rx) options including Monovision. The simulator has a trial frame holder for incorporation of distance sphero-cylindrical refraction correction via trial lenses. In addition, a prism may be added to adjust for the user's interpupillary distance and/or phoria when needed, or the images can be scaled to user's interpupillary distance by reducing the image size and shifting the images outwardly (right eye image to the right, and left eye image to the left) for users with larger interpupillary distance or inwardly for users with a smaller interpupillary distance.

As an example, a local or a web-based software application may be used to run a screening paradigm via the simulator and the display. Additionally, a display microphone and speaker may be used to collect feedback or scores from user related to subjective vision quality or give instructions to the user via the speaker.

As an illustrative example, a multifocal fitting guide may be integrated with a library of images that are convolved by a software model to simulate vision at various distances including distance, intermediate and near. As a further example, the point spread function (PSF) used to convolve the images may be selected to model vision from distance to near with 0.25D steps and up to 2.50D and at various luminance levels including daytime and nighttime scenes. Other steps, ranges, and modeling techniques may be used.

Aspects of the present disclosure may be embodied in a software application. The application may receive inputs such as refractive and biometric data. The input data may comprise one or more of distance optical correction including astigmatism, add needs, eye dominance (or determined via device testing protocol), interpupillary distance, age, gender, previous or habitual correction, pupil size at different luminance levels. Other preference data such as percentage of time spent on distance or near activities may be used.

As described herein, a plurality of images may be generated (e.g., convolved with a point spread function). At least a portion of the images may comprise images that represent a typical real-world experience are selected to be convolved with a point spread function (PSF) that simulates multifocal optics for the user assessment of quality of vision. The scenes may be primarily representative scenes of a near, intermediate or distance object or a combination of such (i.e. a coffee store scene with combined distance, intermediate and/or near scenes). Images may be in color or grayscale and relatable to a user real-word experience. Example experiences may comprise scenes relating to a typical workday, breakfast scene, office scene, night driving, evening television watching, weekend brunch, grocery shopping, reading a book, travelling through an airport, or low light environments. Other images may be used. As an example, at least a portion of the images may be convolved with a PSF that simulates multifocal optics at the appropriate pupil size.

### Image Convolution

As an example, for each optical correction (e.g., low, mid, and high add MFCLs and spherical CLs and IOLs and PALs), point spread functions (PSFs) were computed for object distances (vergences) of −2 to +3.25D in 0.25D steps. PSFs were computed using the Fourier method with a minimum size of 64×64 pixels using a model eye with a spherical aberration of +0.06 D/mm². The chromatic aberration of the eye was assumed to be zero, so that the polychromatic PSFs were constant across the spectrum. PSFs were generated with 4 different pupil diameters, 2.0 and 2.6 mm for near and distance in high luminance (400 cd/m²), and 3.4 and 4.0 mm for near and distance in low luminance (20 cd/m²). The pupil diameter values selected were determined using the reported luminances as inputs to a luminance, age and spherical refraction dependent pupil model (the JJVC pupil model), using an input of Rx=3D and Age=45 years. PSFs were convolved with color images after properly scaling for a 22-degree field of view (FOV) of the display. Note that displaying the images at the proper scale, a scale that matches the scale in the simulation, is required to properly reproduce the magnitude of blur expected with the lenses. Convolutions were performed separately for distance and near parts of scenes. For distance scenes, convolutions were performed for object distances (vergences) of −2.5 to +1.75D, and for near scenes, convolutions were performed for object distances (vergences) of −2.0 to 3.25D. Near scenes included transparency and near objects differed in position between the left and right eye images to impart binocular disparity. When viewed in the stereo-viewer, this disparity created a sense of depth so that the near component of the scene appeared closer to the observer. It is noted that, in a future manifestation, the convolutions could be conducted in real-time permitting use of user specific eye models, either representing averages for user having certain traits (age, Rx, pupil size, accommodative amplitude) or from direct measurements of individual user wavefronts. Also, the PSFs could be adjusted to account for the double-pass nature of the image presentation in an effort to compensate for the fact that the image will be blurred twice, once by the convolution and a second time after passing through the user's optics.

### Image Selection

The images and/or scenes were selected to be:
Relevant to presbyopia—pick everyday real-world scenarios where presbyopes tend to notice their condition.
Engaging—although the images depict everyday scenarios, try to add an element of interest.
Important—what a user tries to see/read matters e.g. the flight number.
Relatable—to life, identity, and culture-try to make the images depict scenes as culturally neutral as possible.

Other metrics and selection criteria may be used.

Various image designs may be used. As a non-limiting example, image design guidelines may include:
Canvas size (66 mm×71.2 mm), or scaled appropriately to the screen size.
Include text with a range of sizes, from around 20/20 to around 20/50.
Only use horizontal text orientation.
Have a combination of both short and long words/sentences/paragraphs.
No motion blur or glare in the images.
‘Not too much going on’ . . . nor too little.
No distorted text angles—it helps to create a ‘flat canvas’ within the image on which to write text e.g. avoid book folds.
Have a combination of low light and high light scenes and reduce contrast slightly in the low light scenes.
Leave some ‘dead space’ between near and far elements to avoid highlighting the lack of need for accommodation.
Generally, use the Optician Sans font (professionally used for Snellen charts).
Use Open Sans font for lower case letters.
20/20 text should not be bolded to prevent it from becoming pixelated.
Make sure the spacing between letters is not too tight (30 pt apart).
Use text and background color with high contrast (although incorporate some variety vs. plain black on white).

Avoid placing text near the bottom corners of the image.

Images may exhibit certain types of correction, including, but not limited to:
Best Distance Sphere, i.e. Single vision correction. An example of a Presbyopic patient that is corrected for distance vision only, including astigmatism, but starts to notice blur symptoms at near vergence.
Best near sphere, i.e. reading glasses. Almost a ⅓ of presbyopic patients utilize reading glasses to address symptoms of presbyopia.
Monovision correction with contact lenses only. Also, a significant proportion of presbyopes are currently corrected with Monovision. This is the case where the dominant eye is corrected for distance and the non-dominant eye is corrected for near vision.
Single vision IOLs and multifocal IOLs along with PALs are other forms of vision correction for presbyopes.

One goal of the incorporation of the above corrections is to ground the patient with the tool, images and their habitual correction in the viewer tool. Scoring of habitual correction image clarity at distance and near will also allow the patient and ECP to compare the MF correction performance to the patient habitual to demonstrate MF performance improvements.

Multifocal and modified monovision correction. Based on patient, distance Rx, and add need, the software then recommends a starting pair of multifocal lenses and retrieves from the image library the correct image.

The system may be programmed to simulate one or more fit guides including the distance and near vision enhancements steps.

The software—based on the add need of the subject—is programmed to select the correct multifocal add lens and to display the corresponding images.

In addition, if the patient scores low on distance image clarity, the operator can choose “distance enhancement” from the drop-down menu and the software is programmed to follow the next steps in the fitting guide. This feature eliminates the potential error in fitting and gives the patient an opportunity to select the correction that is most suitable to them as would be reflected by the image quality score.

During testing, daytime and/or nighttime images are presented to the patient for vision clarity assessment. Each image contains objects at various distances but objects at distance and near are prominent. To ground the patient with each image and with their vision performance, the subject can be probed to read text of various VA equivalent characters on the distance and near image. Then the patient can be asked to score image quality for each by scoring questions from zero (0) to 10 with 10 being very clear and zero (0) being not clear. Other scoring metrics and ranges may be used. The scores may be presented in a performance tracker such as a graphical performance tracker (e.g., spider chart, heptagon performance tracker, or other presentation) to compare habitual to MF correction performance. Other trackers may be used. The questions may include but are not limited to:
Rate the clarity of the daytime distance image.
Rate the clarity of the daytime near image.
Rate the overall image daytime clarity.
Rate the clarity of the nighttime distance image.
Rate the clarity of the nighttime near image.
Rate the overall image nighttime clarity.
Rate the overall vision satisfaction.

### The Testing Paradigm

### User Expectations Management and Conversation Starter

To demonstrate the benefits of an optical correction method, such as multi-focal (MF) lenses, PALs, IOLs, etc., and to allow patients to opt in to considering the optical correction method, the operator may first demonstrate the patient habitual correction, i.e distance Rx, reading glasses and/or monovision. The patient will then score the quality of the vision with the habitual correction.

Then the operator can demonstrate the benefits of the optical correction method to the patient following the recommended fitting guide.

A graphical comparison of habitual vision score(s) vs. vision score(s) associated with the optical correction method can then be presented to the patient showing the performance differences between the two modes of correction.

A patient that is satisfied with their potential vision associated with the optical correction method may elect to initiate conversations about the optical correction method with an ECP and they may decide to try the optical correction method, such as MFCLs, PALs, IOLs, etc.

### Fitting Efficiency

If a patient is not fully satisfied with either their distance and/or near vision clarity, as shown by the graphical score or if the patient verbalizes their dissatisfaction, then a fit assessment can be followed based on a fitting guide (such as a multifocal fitting guide, for example) comprising recommendations for improved distance and/or near vision.

If the patient is then satisfied and considers being fit with the optical correction method (such as MF lenses), then the assessed fit selected can be recommended to the ECP to help increase fit success, reduce chair time and number of revisits to the ECP office.

### Evidence Based Screening

Typically, ECP's screen patients likelihood to be satisfied with vision quality associated with the optical correction method (such as MF lenses) based on the ECP's subjective assessment of patient vision demand. However, the above testing paradigm is more objective as the patient will be judging the quality of their vision prior to committing to trying the optical correction method. As importantly, this can be done without wasting chair time from an ECP perspective. The tool can be operated by staff and/or ECP's with minimal training required.

As an example, a predictive model can be developed based on real world data utilizing the graphical vision scores and patient record of purchasing lenses. The predictive model can then be utilized to predict likelihood of patient satisfaction.

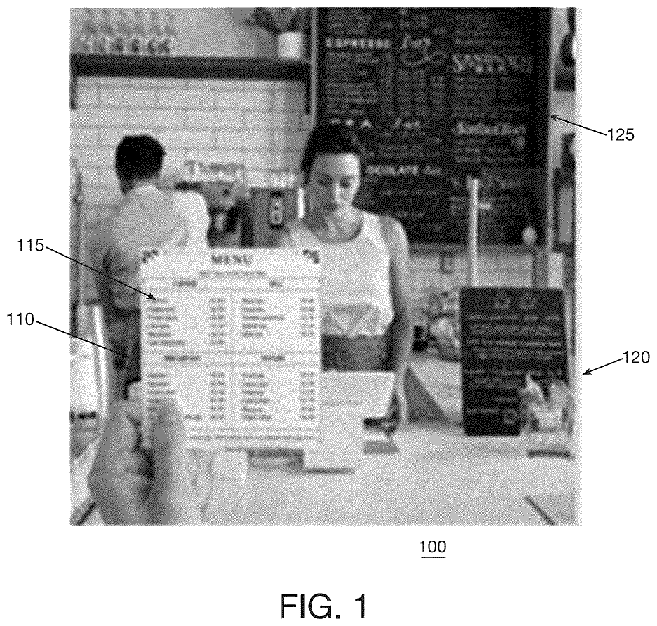

FIG. 1 shows an example image 100 according to the systems and methods described herein. The image may comprise a foreground image 110. The foreground image 110 may comprise a near vision environment. The foreground image 110 may comprise first text 115. The first text 115 may comprise near vision text. For example, the foreground image 110 may comprise an object typically held by a user, such as a holdable menu, a screen of a smart device, a card, a book, a passport, a ticket, etc. The foreground image 110 may comprise an object typically closer to a user when being read, such as a car dashboard, a street sign, etc. Characters in the first text 115 may comprise Optician Sans font. The foreground image 110 may have been made with a foreground reference image. Characters in text of the foreground reference image may be replaced by Optician Sans font in the first text 115 in the foreground image 110. Characters in the first text 115 may correspond to a visual acuity value, such as 20/20, 20/40, etc. A first blurring effect may be applied to the foreground image 110, including the first text 115, to correspond to at least one optical attribute of a contact lens, PAL, IOL, or other vision correction. The at least one optical attribute of a contact lens, multifocal contact lens, PAL, IOL, or other vision correction. may comprise one or more of spherical value, cylindrical value, add power value, or an axis value. Different versions of the foreground image 110 may be made where a blurring effect applied to a version corresponds to at least one optical attribute of the contact lens, PAL, IOL, or other vision correction.

As a non-limiting example, the full through focus PSF (point spread function) may be used as a blur kernel for any depth in a scene. For illustration, a plenoptic camera may capture the full light field of the scene and can therefore infer depth of every pixel. With that information, and using the depth of one or more pixels in an image, one may implement a blur kernel that varies spatially across the image so that it matches the through focus performance for any vision correction and eye model pair.

The image may comprise a background image 120. The background image 120 may comprise a distance vision environment. The background image 120 may comprise second text 125. The second text 125 may comprise distance vision text. For example, the background image 120 may comprise an object typically seen and/or read at distance by a user, such as a menu behind a counter, a screen of a television, a billboard, a highway sign, a listing of flights on a board, etc. Characters in the second text 125 may comprise Optician Sans font. The background image 120 may have been made with a background reference image. Characters in text of the background reference image may be replaced by Optician Sans font in the second text 125 in the background image 120. Characters in the second text 125 may correspond to a visual acuity value, such as 20/20, 20/40, etc. A second blurring effect may be applied to the background image 120, including the second text 125, to correspond to the at least one optical attribute of the contact lens, PAL, IOL, or other vision correction. Different versions of the background image 120 may be made where a blurring effect applied to a version corresponds to at least one optical attribute of the contact lens, PAL, IOL, or other vision correction.

Together, the foreground image 110 and the background image 120 may show a scene. For example, the image 100 may comprise a handheld menu in the foreground image 110 and a café scene with a menu written on a blackboard on a wall in the background image 120. As another example, an image may comprise a boarding pass in a foreground image and a board listing flight number in a background image. The scene depicted by the image 100 may comprise a low luminance environment. The scene depicted by the image 100 may comprise a high luminance environment. Images may be grouped together as a story (theme, journey, etc.). For example, a set of three images may comprise a story associated with vacation. A first image of the set may comprise a high luminance environment of a well-lit airport-showing a boarding pass in a foreground image and a board listing flight number in a background image. A second image of the set may comprise a low luminance environment of beach at dawn-showing a portion of a book in a foreground image and a warning sign in a background image. A third image of the set may comprise a night-time environment showing an illuminated dashboard in a foreground image and a sign for a hotel illuminated by an artificial light in a background image.

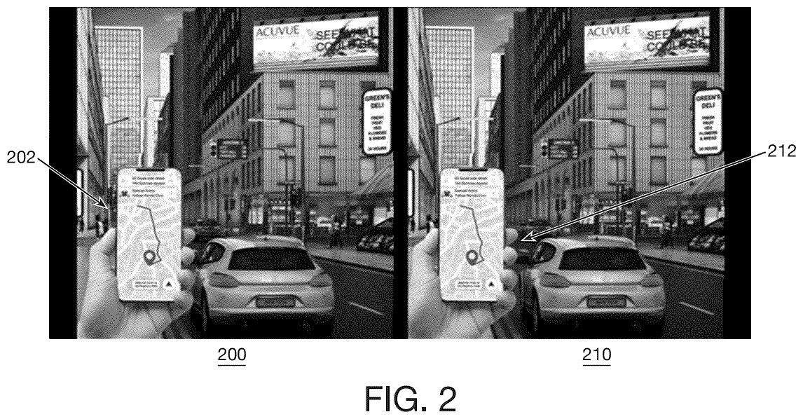

FIG. 2 shows an example left eye image 200 and right eye image 210 pair according to the systems and methods described herein. An image according to the image 100 in FIG. 1 may comprise a left eye image 200 and a right eye image 210 pair. A version of an image according to the image in FIG. 1, wherein a blurring effect applied to the version corresponds to at least one optical attribute of the contact lens, PAL, IOL, or other vision correction, may comprise a left eye image 200 version and a right eye image 210 version pair. The left eye image 200 and the right eye image 210 may be the same. The left eye image 200 and the right eye image 210 may use a same background. The left eye image 200 may use a left eye foreground 202. The right eye image 210 may use a right eye foreground 212. The left eye foreground 202 and the right eye foreground 212 may be the same except that the left eye foreground 202 is shifted horizontally a predetermined number of pixels relative to the right eye foreground 212.

The left eye image 200 may comprise a left eye icon. The right eye image may comprise a right eye icon. When a user uses a device described herein to look at the left eye image with a left eye and the right eye image with a right eye and when the images fuse in the user's vision, the left eye icon and the right eye icon form a merged icon. The left eye icon may comprise a first portion of an icon image. The right eye icon may comprise a second portion of the icon image. The merged icon may comprise the icon image. One of the left eye icon and the right eye icon may comprise a vertical bar; the other of the left eye icon and the right eye icon may comprise a horizontal bar. The merged icon may comprise a vertical line intersecting a horizontal line.

### Systems

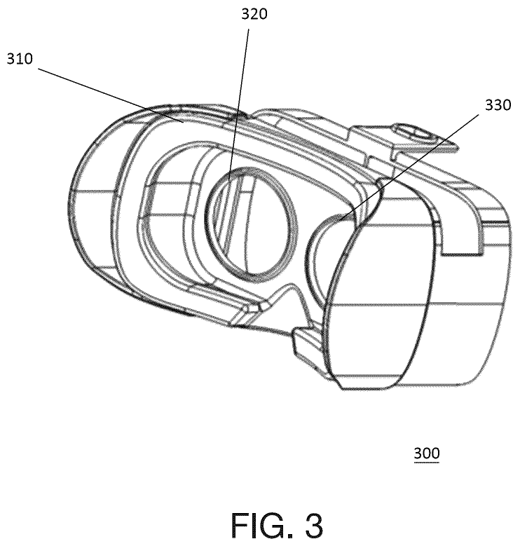

FIG. 3 shows an example head-mount 300 according to the systems and methods described herein. The head-mount 300 may comprise a portion configured to contact a user's face 310. The head-mount 300 may comprise a left eye aperture 320 and a right eye aperture 330. The portion configured to contact a user's face 310 may prevent outside light from entering a user's field of view. The left eye aperture 320 may allow a user to access a left eye image. The right eye aperture 330 may allow a user to access a right eye image.

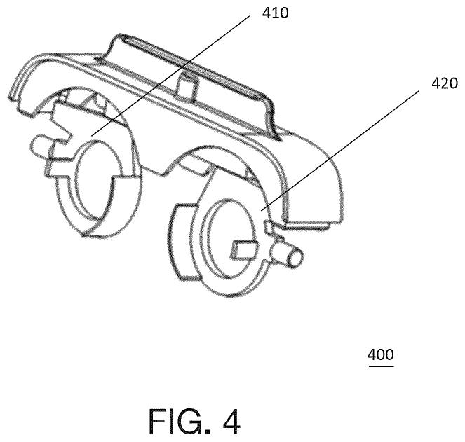

FIG. 4 shows example trial frames 400 according to the systems and methods described herein. The trial frames 400 may comprise a left lens holder 410 and a right lens holder 420. The trial frames 400 may allow for, the use of conventional trial lenses, to correct the user/patient's refractive error (sphere and/or cylinder) and for the viewing distance the images are presented at in the apparatus which represents zero vergence. The trial frames 400 may allow for correction for astigmatism to be applied. For example, frames configured for astigmatic correction may be inserted into the trial frames 400. The trial frames 400 allow for prism lenses to be added. Prism lenses may help maintain a field of view for different inter-pupillary distances and to reduce accommodation-vergence conflict.

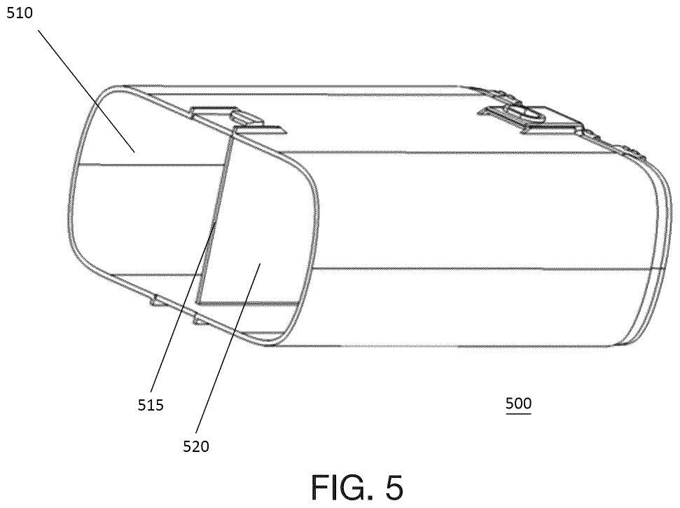

FIG. 5 shows an example tunnel 500 according to the methods and systems described herein. The tunnel 500 may comprise a partition 515 separating a left tunnel portion 510 from a right tunnel portion 520. The tunnel 500 and/or the partition 515 may comprise light-proof and non-reflective material. A user may view a left eye image with a left eye via the left tunnel portion 510. A user may view a right eye image with a right eye via the right tunnel portion 520. The light-proof material may reduce visual distractions for a user. The light-proof material in addition to the +4.00D lens may help a user to relax eyes to infinite focus. Relaxing eyes to infinite focus may help fuse a left eye image and a right eye image into a merged image in a view of a user. Images fuse when a brain of the user mergers two images into a single coherent image. The tunnel 500 may comprise a distance that allows near retinal resolution with a threshold field of view. The tunnel 500 may be about 25 centimeters (cm) in length. The near retinal resolution which is dictated by the resolution of the display may be about 40 cycles per degree. The field of view may be about 16 degrees.

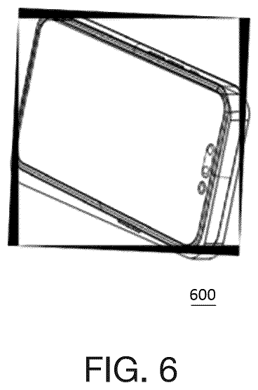

FIG. 6 shows an example display 600 according to the systems and methods described herein. The display 600 may comprise a single screen configured to display a left eye image and a right eye image. The display 600 may comprise two screens, wherein in one screen is configured to display a left eye image and the other screen is configured to display a right eye image. The display 600 may comprise a smartphone. The display 600 may be about 139 millimeters (mm) wide. The display 600 may be about 71.2 mm tall. The display 600 may comprise 2516 pixels horizontally by 1290 pixels vertically. The display 600 may comprise angled pixels, wherein a first portion of the pixels are configured to present a left eye image to a left eye, and wherein a second portion of pixels are configured to present a right eye image to a right eye. The display 600 may be configured to present a left eye image and conceal a right eye image at a first time and present the right eye image and conceal the left eye image at a second time. The display 600 may be configured to present a single image at a time.

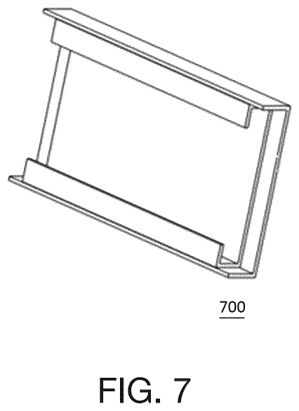

FIG. 7 shows an example display holder 700 according to the systems and methods described herein. The display holder 700 may couple the display 600 to the tunnel 500. The display holder 700 may cause the display 600 to be coupled to the tunnel 500. The display holder 700 may be integrated with the tunnel 500. In an embodiment, the display 600 may be directly integrated with the tunnel 500, eliminating the need for the display holder 700. Other embodiments may be used include a single display or non-stereo embodiment.

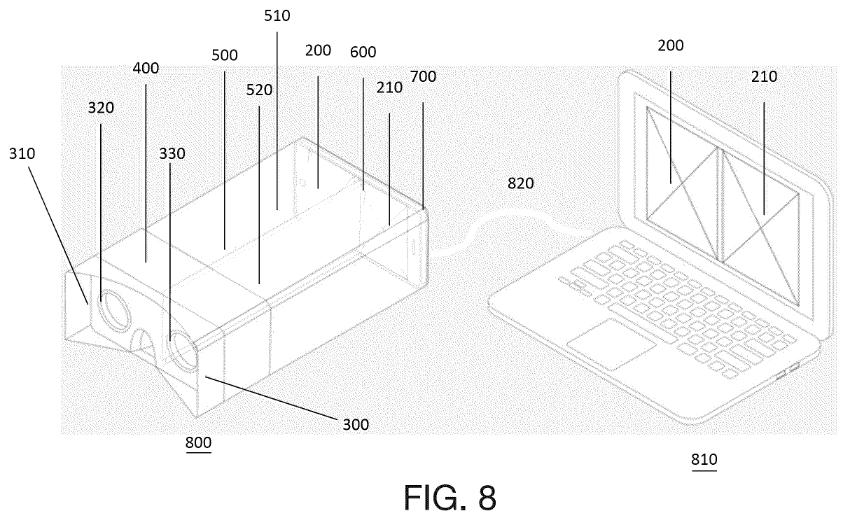

FIG. 8 shows an example device 800 according to the systems and methods described herein. The device 800 may comprise the head-mount 300 described in FIG. 3, the trial frames 400 described in FIG. 4, the tunnel 500 described in FIG. 5, the display 600 described in FIG. 6, and the display holder 700 described in FIG. 7. The device may be handheld by the user/patient or connected to a tripod or stand. The device 800 may be in communication with a computing device 810 via a communication link 820. The computing device 810 may cause a left eye image 200 and a right eye image 210 to be displayed on the display 600. A user may place the device 800 to the head of the user and see the left eye image 200 through the left eye aperture 320 via the left tunnel portion 510 of the tunnel 500 and the right eye image 210 through right eye aperture 330 via the right tunnel portion 520 of the tunnel 520.

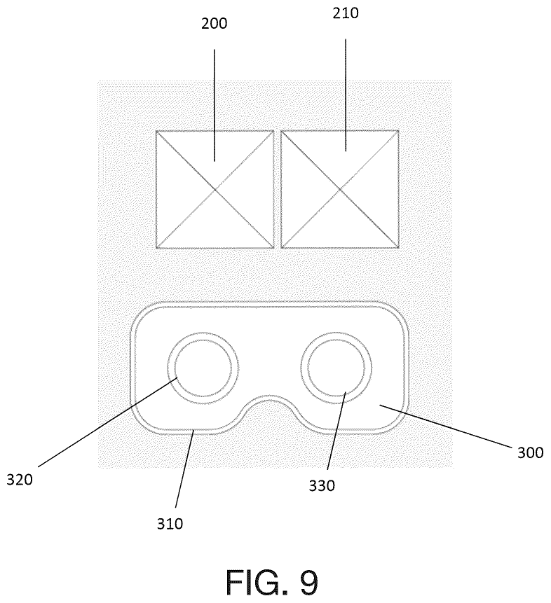

FIG. 9 shows an example front view of the head-mount 300 and an example front view of the left eye image 200 and the right eye image 210. A user may place a face of the user on the portion configured to contact the user's face 310. The user may align a left eye with the left eye aperture 320 and a right eye with the right eye aperture 330. The user may attempt to see the left eye image 200 via the left eye aperture 320 and the right eye image 210 via the right eye aperture 330. If the left eye image 200 and the right eye image 210 do not fuse for a user, then one or more of the left eye image 200 or the right eye image 210 may be adjusted within the display 600. If a need to adjust one or more of the left eye image 200 or the right eye image 210 arises, but an attempted adjustment cannot happen because of a limitation of the display 600, then one or more of the left eye image 200 or the right eye image 210 may be rescaled.

### Methods

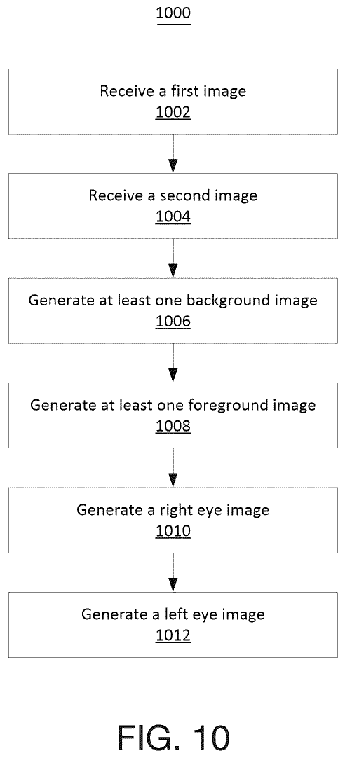

FIG. 10 is a flowchart of an example process 1000. In some implementations, one or more process blocks of FIG. 10 may be performed by the device 800 in FIG. 8, or a computing device, like computing device 810 in FIG. 8.

As shown in FIG. 10, process 1000 may include receiving a first image (block 1002). For example, the device 800 may receive a first image. As another example, the computing device 810 may receive a first image. The first image may have at least a first text. Characters in the first text may be replaced with characters comprising Optician Sans font in the at least one background image.

As shown in FIG. 10, process 1000 may include receiving a second image (block 1004). For example, the device 800 may receive a second image. As another example, the computing device 810 may receive a second image. The second image may have at least a second text. Characters in the second text may be replaced with characters comprising Optician Sans font in the at least one foreground image.

As shown in FIG. 10, process 1000 may include generating at least one background image (block 1006). For example, the device 800 may generate at least one background image. As another example, the computing device 810 may generate at least one background image. The at least one background image may be generated based at least on the first image. The at least one background image may be generated by applying a first blurring effect to at least a portion of the first image. The background image may be generated to emulate a distance vision environment.

As shown in FIG. 10, process 1000 may include generating at least one foreground image (block 1008). For example, the device 800 may generate at least one foreground image. As another example, the computing device 810 may generate at least one foreground image. The at least one foreground image may be generated based at least on the second image. The at least one foreground image may be generated by applying a second blurring effect to at least a portion of the second image. The foreground image may be generated to emulate a near vision environment. The foreground image may emulate near vision in a low luminance situation and the background image may emulate distance vision in the low luminance situation. The foreground image may emulate near vision in a high luminance situation and the background image may emulate distance vision in the high luminance situation.

As shown in FIG. 10, process 1000 may include generating a right eye image (block 1010). For example, the device 800 may generate a right eye image. As another example, the computing device 810 may generate a right eye image. The right eye image may be associated with at least one optical attribute of a contact lens, PAL, IOL, or other vision correction. The right eye image may include at least a first portion of the foreground image overlaying at least a first portion of the background image. The right eye image may be configured to be presented to a right eye of a user. The at least one optical attribute of the contact lens, PAL, IOL, or other vision correction may comprise at least one of spherical value, cylindrical value, add power value, or an axis value.

As shown in FIG. 10, process 1000 may include generating a left eye image (block 1012). For example, the device 800 may generate a left eye image. As another example, the computing device 810 may generate a left eye image. The left eye image may be associated with the at least one optical attribute of the contact lens, PAL, IOL, or other vision correction. The left eye image may include at least a second portion of the foreground image overlaying at least a second portion of the background image. The left eye image may be configured to be presented to a left eye of the user while the right eye image is presented to the right eye of the user. The at least one background image may be the same in the left eye image and the right eye image. The at least one foreground image may be the same in the left eye image and the right eye image. The at least one foreground image in the left eye image may be shifted by a predetermined number of pixels horizontally relative to the at least one foreground image in the right eye image.

The left eye image may comprise a left eye icon. The right eye image may comprise a right eye icon. The left eye icon and the right eye icon may form a merged icon when the user's vision fuses when the user is looking at the left eye image with the left eye and the right eye image with the right eye. Process 1000 may include receiving an indication that that the user's vision did not fuse. For example, the device 800 may receive an indication that that the user's vision did not fuse. As another example, the computing device 810 may receive an indication that that the user's vision did not fuse. Process 1000 may include adjusting at least one of the left eye image or the right eye image horizontally in response to the indication that the user's vision did not fuse. For example, the device 800 may adjust at least one of the left eye image or the right eye image horizontally in response to the indication that the user's vision did not fuse. As another example, the computing device 810 may adjust at least one of the left eye image or the right eye image horizontally in response to the indication that the user's vision did not fuse.

Process 1000 may include receiving an indication that that the user's vision did not fuse. For example the device 800 may receive an indication that that the user's vision did not fuse. As another example, the computing device 810 may receive an indication that that the user's vision did not fuse. Process 1000 may include determining an appropriate adjustment of at least one of the left eye image or the right eye image. For example the device 800 may determine an appropriate adjustment of at least one of the left eye image or the right eye image. As another example, the computing device 810 may determine an appropriate adjustment of at least one of the left eye image or the right eye image. Process 1000 may include determining that at least one of the left eye image or the right eye image is at a boundary. For example, the device 800 may determine that at least one of the left eye image or the right eye image is at a boundary. As another example, the computing device 810 may determine that at least one of the left eye image or the right eye image is at a boundary. The boundary may prevent the appropriate adjustment of the at least one of the left eye image or the right eye image. At least one of the left eye image or the right eye image may be rescaled in response to an indication that adjustment did not cause the user's vision to fuse and at least one of the left eye image or the right eye image cannot be subjected to further adjustment at a current scale.

Process 1000 may include causing the right eye image to be presented to the right eye of the user. For example, the device 800 may cause the right eye image to be presented to the right eye of the user. As another example, the computing device 810 may cause the right eye image to be presented to the right eye of the user. Process 1000 may include causing the left eye image to be presented to the left eye of the user while the right eye image is being presented to the right eye. For example, the device 800 may cause the left eye image to be presented to the left eye of the user while the right eye image is being presented to the right eye. As another example, the computing device 810 may cause the left eye image to be presented to the left eye of the user while the right eye image is being presented to the right eye. The right eye image and the left eye image may be presented via a device comprising a tunnel comprising a first end and a second end. The first end may be configured to be in communication with a face of the user. The second end may be configured to be in communication with at least one display. The first end may comprise a first aperture and a second aperture. The second end may comprise a third aperture and a fourth aperture. The first aperture may be configured to allow the right eye of the user to see the right eye image on the at least one display via the third aperture. The second aperture may be configured to allow the left eye of the user to see the left eye image on the at least one display via the fourth aperture.

Although FIG. 10 shows example blocks of process 1000, in some implementations, process 1000 may include additional blocks, fewer blocks, different blocks, or differently arranged blocks than those depicted in FIG. 10. Additionally, or alternatively, two or more of the blocks of process 1000 may be performed in parallel.

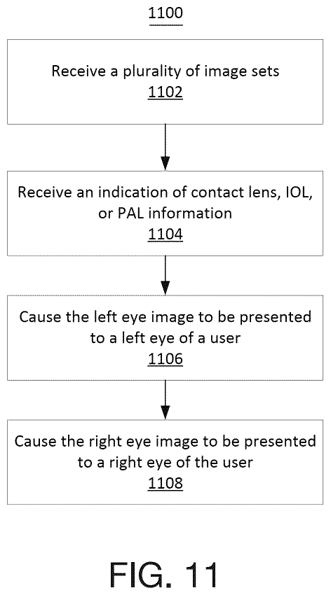

FIG. 11 is a flowchart of an example process 1100. In some implementations, one or more process blocks of FIG. 11 may be performed by the device 800 in FIG. 8, or a computing device, like computing device 810 in FIG. 8.

As shown in FIG. 11, process 1100 may include receiving a plurality of image sets (block 1102). For example, the device 800 may receive a plurality of image sets. As another example, the computing device 810 may receive a plurality of image sets. At least one of the image sets may include a set of versions. At least one of the set of versions may correspond to at least one optical attribute of a contact lens, PAL, IOL, or other vision correction. The at least one of the set of versions may include an image pair. The image pair may include a left eye image and a right eye image. The at least one optical attribute of the contact lens, PAL, IOL, or other vision correction may comprise at least one of spherical value, cylindrical value, add power value, or an axis value.

The left eye image and the right eye image may comprise a background image. The left eye image and the right eye image may comprise a foreground image. The foreground image may emulate near vision in a low luminance situation and the background image may emulate distance vision in the low luminance situation. The foreground image may emulate near vision in a high luminance situation and the background image may emulate distance vision in the high luminance situation. The foreground image in the left eye image may be shifted by a predetermined number of pixels horizontally relative to the foreground image in the right eye image. Characters appearing in the background image and/or the foreground image may be replaced with characters comprising Optician Sans font.

As shown in FIG. 11, process 1100 may include receiving an indication of contact lens, PAL, IOL, or other vision correction information (block 1104). For example, the device 800 may receive an indication of contact lens, PAL, IOL, or other vision correction information. As another example, the computing device 810 may receive an indication of contact lens, PAL, IOL, or other vision correction information. The contact lens, PAL, IOL, or other vision correction information may comprise the at least one optical attribute.

As further shown in FIG. 11, process 1100 may include causing the left eye image to be presented to a left eye of a user (block 1106). For example, the device 800 may cause the left eye image to be presented to a left eye of a user. As another example, the computing device 810 may cause the left eye image to be presented to a left eye of a user.

As also shown in FIG. 11, process 1100 may include causing the right eye image to be presented to a right eye of the user (block 1108). For example, the device 800 may cause the right eye image to be presented to a right eye of the user. As another example, the computing device 810 may cause the right eye image to be presented to a right eye of the user. The left eye image may comprise a left eye icon. The right eye image may comprise a right eye icon. The left eye icon and the right eye icon may form a merged icon when the user's vision fuses when the user is looking at the left eye image with the left eye and the right eye image with the right eye. The left eye icon may comprise a first portion of an icon image. The right eye icon may comprise a second portion of the icon image. The merged icon may comprise the icon image. A first of the left eye icon and the right eye icon may comprise a vertical bar. A second of the left eye icon and the right eye icon may comprise a horizontal bar. The merged icon may comprise a vertical line intersecting a horizontal line.

Although FIG. 11 shows example blocks of process 1100, in some implementations, process 1100 may include additional blocks, fewer blocks, different blocks, or differently arranged blocks than those depicted in FIG. 11. Additionally, or alternatively, two or more of the blocks of process 1100 may be performed in parallel.

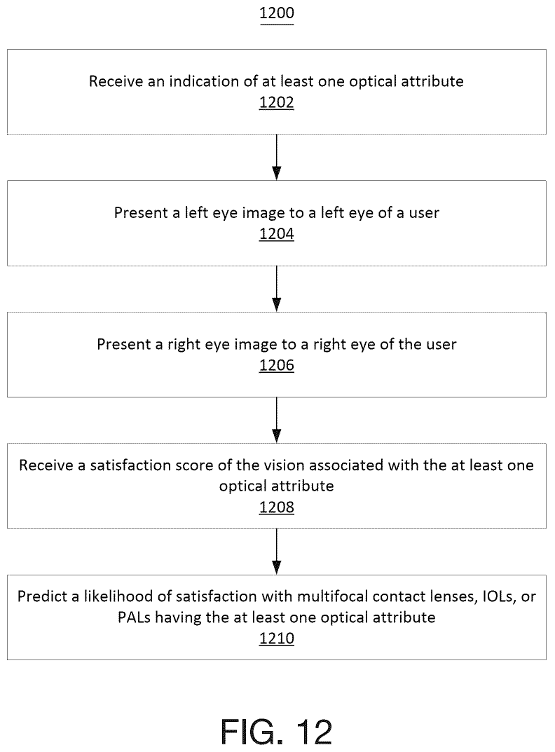

FIG. 12 is a flowchart of an example process 1200. In some implementations, one or more process blocks of FIG. 12 may be performed by the device 800 in FIG. 8, or a computing device, like computing device 810 in FIG. 8.

As shown in FIG. 12, process 1200 may include receiving an indication of at least one optical attribute (block 1202). For example, the device 800 may receive an indication of at least one optical attribute. As another example, the computing device 810 may receive an indication of at least one optical attribute. The at least one optical attribute may comprise at least one of spherical value, cylindrical value, add power value, or an axis value.

As shown in FIG. 12, process 1200 may include presenting a left eye image to a left eye of a user (block 1204). For example, the device 800 may present a left eye image to a left eye of a user. As another example, the computing device 810 may present a left eye image to a left eye of a user. The left eye image may emulate vision associated with the at least one optical attribute.

As shown in FIG. 12, process 1200 may include presenting a right eye image to a right eye of the user (block 1206). For example, the device 800 may present a right eye image to a right eye of the user. As another example, the computing device 810 may present a right eye image to a right eye of the user. The right eye image may emulate the vision associated with the at least one optical attribute.

As shown in FIG. 12, process 1200 may include receiving a satisfaction score of the vision associated with the at least one optical attribute (block 1208). For example, the device 800 may receive a satisfaction score of the vision associated with the at least one optical attribute. As another example, the computing device 810 may receive a satisfaction score of the vision associated with the at least one optical attribute. The satisfaction score may be received from the user. The left eye image and the right eye image may comprise a background image and a foreground image. The satisfaction score may be based on at least a satisfaction with near vision based on the foreground image and a satisfaction with distance vision based on the background image.

As shown in FIG. 12, process 1200 may include predicting a likelihood of satisfaction with multifocal contact lenses, PAL, IOL, or other vision correction having the at least one optical attribute (block 1210). For example, the device 800 may predict a likelihood of satisfaction with multifocal contact lenses, PAL, IOL, or other vision correction having the at least one optical attribute. As another example, the computing device 810 may predict a likelihood of satisfaction with multifocal contact lenses, PAL, IOL, or other vision correction having the at least one optical attribute. The predicting a likelihood of satisfaction with multifocal contact lenses, PAL, IOL, or other vision correction having the at least one optical attribute may be based on the satisfaction score. The predicting a likelihood of satisfaction with multifocal contact lenses, PAL, IOL, or other vision correction comprising the at least one optical attribute based on the satisfaction score may comprise using a model trained on previous satisfaction scores and previous assessments of satisfaction with contact lenses, PALs, IOLs, or other vision corrections.

Although FIG. 12 shows example blocks of process 1200, in some implementations, process 1200 may include additional blocks, fewer blocks, different blocks, or differently arranged blocks than those depicted in FIG. 12. Additionally, or alternatively, two or more of the blocks of process 1200 may be performed in parallel.

### EXAMPLE CLAUSES

### Example Clause 1

A method may include: receiving a first image may include at least a first text; receiving a second image may include at least a second text; generating, based at least on the first image, at least one background image by applying a first blurring effect to at least a portion of the first image, where the background image is generated to emulate a distance vision environment; generating, based at least on the second image, at least one foreground image by applying a second blurring effect to at least a portion of the second image, where the foreground image is generated to emulate a near vision environment; generating a right eye image associated with at least one attribute of a contact lens, where the right eye image may include at least a first portion of the foreground image overlaying at least a first portion of the background image, where the right eye image is configured to be presented to a right eye of a user; and generating a left eye image associated with the at least one attribute of the contact lens, where the left eye image may include at least a second portion of the foreground image overlaying at least a second portion of the background image, where the left eye image is configured to be presented to a left eye of the user while the right eye image is presented to the right eye of the user.

### Example Clause 2

The method of Example Clause 1, further may include: causing the right eye image to be presented to the right eye of the user; and causing the left eye image to be presented to the left eye of the user while the right eye image is being presented to the right eye.

### Example Clause 3

The method of Example Clause 1 or Example Clause 2, where the right eye image and the left eye image are presented via a device may include a tunnel may include a first end and a second end, where the first end is configured to be in communication with a face of the user, where the second end is configured to be in communication with at least one display, where the first end may include a first aperture and a second aperture, where the second end may include a third aperture and a fourth aperture, where the first aperture is configured to allow the right eye of the user to see the right eye image on the at least one display via the third aperture, and where the second aperture is configured to allow the left eye of the user to see the left eye image on the at least one display via the fourth aperture.

### Example Clause 4

The method of any one of Example Clauses 1-3, where characters in the first text are replaced with characters may include Optician Sans font in the at least one background image.

### Example Clause 5

The method of any one of Example Clauses 1-4, where characters in the second text are replaced with characters may include Optician Sans font in the at least one foreground image.

### Example Clause 6

The method of any one of Example Clauses 1-5, where the at least one background image is the same in the left eye image and the right eye image.

### Example Clause 7

The method of any one of Example Clauses 1-6, where the at least one foreground image is the same in the left eye image and the right eye image.

### Example Clause 8

The method of any one of Example Clauses 1-7, where the at least one foreground image in the left eye image is shifted by a predetermined number of pixels horizontally relative to the at least one foreground image in the right eye image.

### Example Clause 9

The method of any one of Example Clauses 1-8, where the left eye image may include a left eye icon, where the right eye image may include a right eye icon, and where the left eye icon and the right eye icon form a merged icon when the user's vision fuses when the user is looking at the left eye image with the left eye and the right eye image with the right eye.

### Example Clause 10

The method of any one of Example Clauses 1-9, further may include: receiving an indication that that the user's vision did not fuse; and adjusting at least one of the left eye image or the right eye image horizontally in response to the indication that the user's vision did not fuse.

### Example Clause 11

The method of any one of Example Clauses 1-10, further may include: receiving an indication that that the user's vision did not fuse; determining an appropriate adjustment of at least one of the left eye image or the right eye image; determining that at least one of the left eye image or the right eye image is at a boundary, where the boundary prevents the appropriate adjustment of the at least one of the left eye image or the right eye image; and where at least one of the left eye image or the right eye image are rescaled in response to an indication that adjustment did not cause the user's vision to fuse and at least one of the left eye image or the right eye image cannot be subjected to further adjustment at a current scale.

### Example Clause 12

The method of any one of Example Clauses 1-11, where the foreground image emulates near vision in a low luminance situation and the background image emulates distance vision in the low luminance situation.

### Example Clause 13

The method of any one of Example Clauses 1-12, where the foreground image emulates near vision in a high luminance situation and the background image emulates distance vision in the high luminance situation.

### Example Clause 14

The method of any one of Example Clauses 1-13, where the at least one attribute of the contact lens may include at least one of spherical value, cylindrical value, add power value, or an axis value.

### Example Clause 15

A method may include: receiving a plurality of image sets, where at least one of the image sets may include a set of versions, where at least one of the set of versions corresponds to at least one attribute of a contact lens, where the at least one of the set of versions may include an image pair, and where the image pair may include a left eye image and a right eye image; receiving an indication of contact lens information, where the contact lens information may include the at least one attribute; causing the left eye image to be presented to a left eye of a user; and causing the right eye image to be presented to a right eye of the user.

### Example Clause 16

The method of Example Clause 15, where the left eye image and the right eye image may include a background image, and where the left eye image and the right eye image may include a foreground image.

### Example Clause 17

The method of Example Clause 15 or Example Clause 16, where the foreground image emulates near vision in a low luminance situation and the background image emulates distance vision in the low luminance situation.

### Example Clause 18

The method of any one of Example Clauses 15-17, where the foreground image emulates near vision in a high luminance situation and the background image emulates distance vision in the high luminance situation.

### Example Clause 19

The method of any one of Example Clauses 15-18, where the foreground image in the left eye image is shifted by a predetermined number of pixels horizontally relative to the foreground image in the right eye image.

### Example Clause 20

The method of any one of Example Clauses 15-19, where characters appearing in the background image and/or the foreground image are replaced with characters may include Optician Sans font.

### Example Clause 21

The method of any one of Example Clauses 15-20, where the left eye image may include a left eye icon, where the right eye image may include a right eye icon, and where the left eye icon and the right eye icon form a merged icon when the user's vision fuses when the user is looking at the left eye image with the left eye and the right eye image with the right eye.

### Example Clause 22

The method of any one of Example Clauses 15-21, where the left eye icon may include a first portion of an icon image, where the right eye icon may include a second portion of the icon image, and where the merged icon may include the icon image.

### Example Clause 23

The method of any one of Example Clauses 15-22, where a first of the left eye icon and the right eye icon may include a vertical bar, where a second of the left eye icon and the right eye icon may include a horizontal bar, and where the merged icon may include a vertical line intersecting a horizontal line.

### Example Clause 24

The method of any one of Example Clauses 15-23, where the at least one attribute of the contact lens may include at least one of spherical value, cylindrical value, add power value, or an axis value.

### Example Clause 25

A method may include: receiving an indication of at least one multifocal contact lens attribute; presenting a left eye image to a left eye of a user, where the left eye image emulates vision associated with the at least one multifocal contact lens attribute; presenting a right eye image to a right eye of the user, where the right eye image emulates the vision associated with the at least one multifocal contact lens attribute; receiving a satisfaction score of the vision associated with the at least one multifocal contact lens attribute from the user; and predicting a likelihood of satisfaction with multifocal contact lenses may include the at least one multifocal contact lens attribute based on the satisfaction score.

### Example Clause 26

The method of Example Clause 25, where the left eye image and the right eye image may include a background image and a foreground image, and where the satisfaction score is based on at least a satisfaction with near vision based on the foreground image and a satisfaction with distance vision based on the background image.

### Example Clause 27

The method of Example Clause 25 or Example Clause 26, where the predicting a likelihood of satisfaction with multifocal contact lenses may include the at least one multifocal contact lens attribute based on the satisfaction score may include using a model trained on previous satisfaction scores and previous assessments of satisfaction with contact lenses.

### Example Clause 28

The method of any one of Example Clauses 25-27, where the at least one multifocal contact lens attribute may include at least one of spherical value, cylindrical value, add power value, or an axis value.

### Example Clause 29

A system may include: one or more processors configured to: receive a first image may include at least a first text; receive a second image may include at least a second text; generate, based at least on the first image, at least one background image by applying a first blurring effect to at least a portion of the first image, where the background image is generated to emulate a distance vision environment; generate, based at least on the second image, at least one foreground image by applying a second blurring effect to at least a portion of the second image, where the foreground image is generated to emulate a near vision environment; generate a right eye image associated with at least one attribute of a contact lens, where the right eye image may include at least a first portion of the foreground image overlaying at least a first portion of the background image, where the right eye image is configured to be presented to a right eye of a user; and generate a left eye image associated with the at least one attribute of the contact lens, where the left eye image may include at least a second portion of the foreground image overlaying at least a second portion of the background image, where the left eye image is configured to be presented to a left eye of the user while the right eye image is presented to the right eye of the user.

### Example Clause 30

The system of Example Clause 29, where the one or more processors are further configured to: causing the right eye image to be presented to the right eye of the user; and causing the left eye image to be presented to the left eye of the user while the right eye image is being presented to the right eye.

### Example Clause 31

The system of Example Clause 29 or Example Clause 30, where the right eye image and the left eye image are presented via a device may include a tunnel may include a first end and a second end, where the first end is configured to be in communication with a face of the user, where the second end is configured to be in communication with at least one display, where the first end may include a first aperture and a second aperture, where the second end may include a third aperture and a fourth aperture, where the first aperture is configured to allow the right eye of the user to see the right eye image on the at least one display via the third aperture, and where the second aperture is configured to allow the left eye of the user to see the left eye image on the at least one display via the fourth aperture.

### Example Clause 32

The system of any one of Example Clauses 29-31, where characters in the first text are replaced with characters may include Optician Sans font in the at least one background image.

### Example Clause 33

The system of any one of Example Clauses 29-32, where characters in the second text are replaced with characters may include Optician Sans font in the at least one foreground image.

### Example Clause 34

The system of any one of Example Clauses 29-33, where the at least one background image is the same in the left eye image and the right eye image.

### Example Clause 35

The system of any one of Example Clauses 29-34, where the at least one foreground image is the same in the left eye image and the right eye image.

### Example Clause 36

The system of any one of Example Clauses 29-35, where the at least one foreground image in the left eye image is shifted by a predetermined number of pixels horizontally relative to the at least one foreground image in the right eye image.

### Example Clause 37

The system of any one of Example Clauses 29-36, where the left eye image may include a left eye icon, where the right eye image may include a right eye icon, and where the left eye icon and the right eye icon form a merged icon when the user's vision fuses when the user is looking at the left eye image with the left eye and the right eye image with the right eye.

### Example Clause 38

The system of any one of Example Clauses 29-37, where the one or more processors are further configured to: receiving an indication that that the user's vision did not fuse; and adjusting at least one of the left eye image or the right eye image horizontally in response to the indication that the user's vision did not fuse.

### Example Clause 39

The system of any one of Example Clauses 29-38, where the one or more processors are further configured to: receiving an indication that that the user's vision did not fuse; determining an appropriate adjustment of at least one of the left eye image or the right eye image; determining that at least one of the left eye image or the right eye image is at a boundary, where the boundary prevents the appropriate adjustment of the at least one of the left eye image or the right eye image; and where at least one of the left eye image or the right eye image are rescaled in response to an indication that adjustment did not cause the user's vision to fuse and at least one of the left eye image or the right eye image cannot be subjected to further adjustment at a current scale.

### Example Clause 40

The system of any one of Example Clauses 29-39, where the foreground image emulates near vision in a low luminance situation and the background image emulates distance vision in the low luminance situation.

### Example Clause 41

The system of any one of Example Clauses 29-40, where the foreground image emulates near vision in a high luminance situation and the background image emulates distance vision in the high luminance situation.

### Example Clause 42

The system of any one of Example Clauses 29-41, where the at least one attribute of the contact lens may include at least one of spherical value, cylindrical value, add power value, or an axis value.

### Example Clause 43

A system may include: one or more processors configured to: receive a plurality of image sets, where at least one of the image sets may include a set of versions, where at least one of the set of versions corresponds to at least one attribute of a contact lens, where the at least one of the set of versions may include an image pair, and where the image pair may include a left eye image and a right eye image; receive an indication of contact lens information, where the contact lens information may include the at least one attribute; cause the left eye image to be presented to a left eye of a user; and cause the right eye image to be presented to a right eye of the user.

### Example Clause 44

The system of Example Clause 43, where the left eye image and the right eye image may include a background image, and where the left eye image and the right eye image may include a foreground image.

### Example Clause 45

The system of Example Clause 43 or Example Clause 44, where the foreground image emulates near vision in a low luminance situation and the background image emulates distance vision in the low luminance situation.

### Example Clause 46

The system of any one of Example Clauses 43-45, where the foreground image emulates near vision in a high luminance situation and the background image emulates distance vision in the high luminance situation.

### Example Clause 47

The system of any one of Example Clauses 43-46, where the foreground image in the left eye image is shifted by a predetermined number of pixels horizontally relative to the foreground image in the right eye image.

### Example Clause 48

The system of any one of Example Clauses 43-47, where characters appearing in the background image and/or the foreground image are replaced with characters may include Optician Sans font.

### Example Clause 49

The system of any one of Example Clauses 43-48, where the left eye image may include a left eye icon, where the right eye image may include a right eye icon, and where the left eye icon and the right eye icon form a merged icon when the user's vision fuses when the user is looking at the left eye image with the left eye and the right eye image with the right eye.

### Example Clause 50

The system of any one of Example Clauses 43-49, where the left eye icon may include a first portion of an icon image, where the right eye icon may include a second portion of the icon image, and where the merged icon may include the icon image.

### Example Clause 51

The system of any one of Example Clauses 43-50, where a first of the left eye icon and the right eye icon may include a vertical bar, where a second of the left eye icon and the right eye icon may include a horizontal bar, and where the merged icon may include a vertical line intersecting a horizontal line.

### Example Clause 52

The system of any one of Example Clauses 43-51, where the at least one attribute of the contact lens may include at least one of spherical value, cylindrical value, add power value, or an axis value.

### Example Clause 53

A system may include: one or more processors configured to: receive an indication of at least one multifocal contact lens attribute; present a left eye image to a left eye of a user, where the left eye image emulates vision associated with the at least one multifocal contact lens attribute; present a right eye image to a right eye of the user, where the right eye image emulates the vision associated with the at least one multifocal contact lens attribute; receive a satisfaction score of the vision associated with the at least one multifocal contact lens attribute from the user; and predict a likelihood of satisfaction with multifocal contact lenses may include the at least one multifocal contact lens attribute based on the satisfaction score.

### Example Clause 54

The system of Example Clause 53, where the left eye image and the right eye image may include a background image and a foreground image, and where the satisfaction score is based on at least a satisfaction with near vision based on the foreground image and a satisfaction with distance vision based on the background image.

### Example Clause 55

The system of Example Clause 53 or Example Clause 54, where the predicting a likelihood of satisfaction with multifocal contact lenses may include the at least one multifocal contact lens attribute based on the satisfaction score may include using a model trained on previous satisfaction scores and previous assessments of satisfaction with contact lenses.

### Example Clause 56

The system of any one of Example Clauses 53-55, where the at least one multifocal contact lens attribute may include at least one of spherical value, cylindrical value, add power value, or an axis value.

The foregoing disclosure provides illustration and description but is not intended to be exhaustive or to limit the implementations to the precise form disclosed. Modifications may be made in light of the above disclosure or may be acquired from practice of the implementations. As used herein, the term “component” is intended to be broadly construed as hardware, firmware, or a combination of hardware and software. It will be apparent that systems and/or methods described herein may be implemented in different forms of hardware, firmware, and/or a combination of hardware and software. The actual specialized control hardware or software code used to implement these systems and/or methods is not limiting of the implementations. Thus, the operation and behavior of the systems and/or methods are described herein without reference to specific software code-it being understood that software and hardware can be used to implement the systems and/or methods based on the description herein. As used herein, satisfying a threshold may, depending on the context, refer to a value being greater than the threshold, greater than or equal to the threshold, less than the threshold, less than or equal to the threshold, equal to the threshold, and/or the like, depending on the context. Although particular combinations of features are recited in the claims and/or disclosed in the specification, these combinations are not intended to limit the disclosure of various implementations. In fact, many of these features may be combined in ways not specifically recited in the claims and/or disclosed in the specification

Although each dependent claim listed below may directly depend on only one claim, the disclosure of various implementations includes each dependent claim in combination with every other claim in the claim set. No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, a combination of related and unrelated items, and/or the like), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

1-28. (canceled)

29. A system comprising:
one or more processors configured to:
receive a first image;
receive a second image;
generate, based at least on the first image, at least one background image by applying a first blurring effect to at least a portion of the first image, wherein the background image is generated to emulate a distance vision environment;
generate, based at least on the second image, at least one foreground image by applying a second blurring effect to at least a portion of the second image, wherein the foreground image is generated to emulate a near vision environment;
generate a right eye image associated with at least one optical attribute of a contact lens, interocular lens (IOL), or progressive addition lens (PAL), wherein the right eye image comprises at least a first portion of the foreground image overlaying at least a first portion of the background image, wherein the right eye image is configured to be presented to a right eye of a user; and
generate a left eye image associated with the at least one optical attribute of the contact lens, IOL, or PAL, wherein the left eye image comprises at least a second portion of the foreground image overlaying at least a second portion of the background image, wherein the left eye image is configured to be presented to a left eye of the user while the right eye image is presented to the right eye of the user.

30. The system of claim 29, wherein the one or more processors are further configured to:
causing the right eye image to be presented to the right eye of the user; and
causing the left eye image to be presented to the left eye of the user while the right eye image is being presented to the right eye.

31. The system of claim 30, wherein the right eye image and the left eye image are presented via a device comprising a tunnel comprising a first end and a second end, wherein the first end is configured to be in communication with a face of the user, wherein the second end is configured to be in communication with at least one display, wherein the first end comprises a first aperture and a second aperture, wherein the second end comprises a third aperture and a fourth aperture, wherein the first aperture is configured to allow the right eye of the user to see the right eye image on the at least one display via the third aperture, and wherein the second aperture is configured to allow the left eye of the user to see the left eye image on the at least one display via the fourth aperture.

32. The system of claim 29, wherein the first images comprises a first text or comprises Optician Sans font in the at least one background image.

33. The system of claim 29, wherein the second image comprises a second text or comprises Optician Sans font in the at least one foreground image.

34. The system of claim 29, wherein the at least one background image is the same in the left eye image and the right eye image.

35. The system of claim 34, wherein the at least one foreground image is the same in the left eye image and the right eye image.

36. The system of claim 35, wherein the at least one foreground image in the left eye image is shifted by a predetermined number of pixels horizontally relative to the at least one foreground image in the right eye image.

37. The system of claim 29, wherein the left eye image comprises a left eye icon, wherein the right eye image comprises a right eye icon, and wherein the left eye icon and the right eye icon form a merged icon when the user's vision fuses when the user is looking at the left eye image with the left eye and the right eye image with the right eye.

38. The system of claim 37, wherein the one or more processors are further configured to:
receiving an indication that that the user's vision did not fuse; and
adjusting at least one of the left eye image or the right eye image horizontally in response to the indication that the user's vision did not fuse.

39. The system of claim 37, wherein the one or more processors are further configured to:
receiving an indication that that the user's vision did not fuse;
determining an appropriate adjustment of at least one of the left eye image or the right eye image;
determining that at least one of the left eye image or the right eye image is at a boundary, wherein the boundary prevents the appropriate adjustment of the at least one of the left eye image or the right eye image; and
wherein at least one of the left eye image or the right eye image are rescaled in response to an indication that adjustment did not cause the user's vision to fuse and at least one of the left eye image or the right eye image cannot be subjected to further adjustment at a current scale.

40. The system of claim 29, wherein the foreground image emulates near vision in a low luminance situation and the background image emulates distance vision in the low luminance situation.

41. The system of claim 29, wherein the foreground image emulates near vision in a high luminance situation and the background image emulates distance vision in the high luminance situation.

42. The system of claim 29, wherein the at least one optical attribute of the contact lens, IOL, or PAL comprises at least one of spherical value, cylindrical value, add power value, or an axis value.

43-56. (canceled)
